SEQUENCE LISTING (7 sequences)

SEQ ID NO: 1 - DNA, 1380 bp, Arabidopsis thaliana
atgggaagtatcgtggagccatgggcagcaatcgccgagaacggaaacgcaaacgtgaccgcgaaaggctcgagcagggagctgcgacatgggagaacagcacacaacatgtcttcatcgtcgctaaggaagaaatcagacctgagattggttcagaaagttccatgcaaaactctcaagaacattctctctaatcttcaagaagtcattcttggtactaagcttactctcttatttctcgccatccctctcgccattcttgccaattcttacaactacggtcgtccgttgatatttggactgagcttgatagggctgacacctctagctgagcgagttagctttttgacagagcaactagctttctacactggtccaacagtgggcggtttgttgaacgcgacttgtggaaacgcgacagagctgataatcgcgatactagcgttggccaataacaaagtggcagtggtgaaatactctctattgggttcaattctctcaaaccttctcttggttcttggcacttccctcttctttggtggtatcgccaatatccgccgcgagcagcggttcgaccggaaacaagccgatgtgaacttcttcttgctgcttatgggcctgttgtgtcatttgctgccattattgttaaaatatgcagcaaccggcgaagtatcgacctctatgattaacaaaatgtcgctcactctgtcgcggacaagcagcatagttatgcttattgcttacattgcttatctcatcttccagctctggactcaccgccaattgtttgaggcacaacaggatgatgatgatgcatatgatgatgaggttagtgttgaagaaactccagtgataggattctggagcggatttgcttggctcgttgggatgacaatagtcatcgcattgctatcagagtatgttgtggacacgatcgaggatgcatcggactcatggggactatcagtgagtttcataagcatcatattgcttcccattgttgggaatgcggctgagcatgctggagccatcattttcgcattcaagaacaagctcgacatatctctaggggttgcgttgggctctgcaactcagatttctttgttcgtggtcccattgagtgttatcgttgcgtggatcctgggaataaaaatggatctcaactttaacatccttgaaactagctctctagctttggccattatcatcacagccttcactttacaggatggaacttctcattacatgaagggactggttctattgttatgctatgtcatcatcgcggcgtgtttcttcgtcgaccaaattccccaaccaaatgatttggacgtgggacttcaacccatgaacaatttgggagaagttttctcagcttaa

SEQ ID NO: 2 - protein, 459 aa, Arabidopsis thaliana
MGSIVEPWAAIAENGNANVTAKGSSRELRHGRTAHNMSSSSLRKKSDLRLVQKVPCKTLKNILSNLQEVILGTKLTLLFLAIPLAILANSYNYGRPLIFGLSLIGLTPLAERVSFLTEQLAFYTGPTVGGLLNATCGNATELIIAILALANNKVAVVKYSLLGSILSNLLLVLGTSLFFGGIANIRREQRFDRKQADVNFFLLLMGLLCHLLPLLLKYAATGEVSTSMINKMSLTLSRTSSIVMLIAYIAYLIFQLWTHRQLFEAQQDDDDAYDDEVSVEETPVIGFWSGFAWLVGMTIVIALLSEYVVDTIEDASDSWGLSVSFISIILLPIVGNAAEHAGAIIFAFKNKLDISLGVALGSATQISLFVVPLSVIVAWILGIKMDLNFNILETSSLALAIIITAFTLQDGTSHYMKGLVLLLCYVIIAACFFVDQIPQPNDLDVGLQPMNNLGEVFSA

SEQ ID NO: 3 - DNA, 1236 bp, Arabidopsis thaliana
atgtgcaaaactctcaagaacattctctctaatcttcaagaagtcattcttggtactaagcttactctcttatttctcgccatccctctcgccattcttgccaattcttacaactacggtcgtccgttgatatttggactgagcttgatagggctgacacctctagctgagcgagttagctttttgacagagcaactagctttctacactggtccaacagtgggcggtttgttgaacgcgacttgtggaaacgcgacagagctgataatcgcgatactagcgttggccaataacaaagtggcagtggtgaaatactctctattgggttcaattctctcaaaccttctcttggttcttggcacttccctcttctttggtggtatcgccaatatccgccgcgagcagcggttcgaccggaaacaagccgatgtgaacttcttcttgctgcttatgggcctgttgtgtcatttgctgccattattgttaaaatatgcagcaaccggcgaagtatcgacctctatgattaacaaaatgtcgctcactctgtcgcggacaagcagcatagttatgcttattgcttacattgcttatctcatcttccagctctggactcaccgccaattgtttgaggcacaacaggatgatgatgatgcatatgatgatgaggttagtgttgaagaaactccagtgataggattctggagcggatttgcttggctcgttgggatgacaatagtcatcgcattgctatcagagtatgttgtggacacgatcgaggatgcatcggactcatggggactatcagtgagtttcataagcatcatattgcttcccattgttgggaatgcggctgagcatgctggagccatcattttcgcattcaagaacaagctcgacatatctctaggggttgcgttgggctctgcaactcagatttctttgttcgtggtcccattgagtgttatcgttgcgtggatcctgggaataaaaatggatctcaactttaacatccttgaaactagctctctagctttggccattatcatcacagccttcactttacaggatggaacttctcattacatgaagggactggttctattgttatgctatgtcatcatcgcggcgtgtttcttcgtcgaccaaattccccaaccaaatgatttggacgtgggacttcaacccatgaacaatttgggagaagttttctcagctcatcatcatcatcatcattaa

SEQ ID NO: 4 - protein, 411 aa, Arabidopsis thaliana
MCKTLKNILSNLQEVILGTKLTLLFLAIPLAILANSYNYGRPLIFGLSLIGLTPLAERVSFLTEQLAFYTGPTVGGLLNATCGNATELIIAILALANNKVAVVKYSLLGSILSNLLLVLGTSLFFGGIANIRREQRFDRKQADVNFFLLLMGLLCHLLPLLLKYAATGEVSTSMINKMSLTLSRTSSIVMLIAYIAYLIFQLWTHRQLFEAQQDDDDAYDDEVSVEETPVIGFWSGFAWLVGMTIVIALLSEYVVDTIEDASDSWGLSVSFISIILLPIVGNAAEHAGAIIFAFKNKLDISLGVALGSATQISLFVVPLSVIVAWILGIKMDLNFNILETSSLALAIIITAFTLQDGTSHYMKGLVLLLCYVIIAACFFVDQIPQPNDLDVGLQPMNNLGEVFSAHHHHHH

SEQ ID NO: 5 - protein, 444 aa, Vigna radiata
MGSHQHEPWLLENGNPKVLTREMRHGRTAHSKSSNSLRTKSDRTLVSKVPCATIRNVLFNLQEVILGTKLSILIPAIPVAIVAEYCGFGRPWVFVLSLLGLTPLAERVSFITEQVAFYTGPTVGGLLNATCGNVTELIIAIFALSSNKIAVVKYSLLGSILSNLLLVLGTSLLCGGIANVGLEQKYDRRQGDVNSLMLLLALLCYLLPMLFKYSAASAALTVDPSLHLSRASSIVMLIAYVVYIIFQLWTHRQLFEAEDEDEDDNNGSDEQAVIGLWSGIAWLIGMTVFIALLSEYVVDTIEDASDSWGLSVSFLSIILLPIVGNAAEHAGAVIFAFKNKLDISLGVALGSATQIAMFVVPLCVIVAWTMGVKMDLNFNILETGSVALAIIVTSFTLQDGTSHYMKGLVLLLCYIVIGACFFVQRTPFNQADVTNVIPNGVLSA

SEQ ID NO: 6 - protein, 424 aa, Oryza sativa
MSSSSLRKKSDAALVRKVPVAPLRPLLANLQEVFLATKLAVLFPAVPLAIAAQCFRFDQVWVFALSLLGLIPLAERVSFLTEQIALYTGPTVGGLLNATCGNATELIIALFALLKGKIEVVKCSLLGSVLSNLLLVLGTSLFCGGVVNLGARQPYDRNQSDVSTALLFLAVLCHSAPLLLRYAVAAGEHSVSATSAAASLDLSRACSFVMLASYVAYLFFQLKTHRQLFEPQEVDGGDAGDDDEEPALGFASALFWLALMTAVISVLSEYVVGTIEPTSQSWGLSVSFISIILLPIVGNAAEHAGAIIFALKNKLDITLGVALGSATQISMFVVPLSVLVAWIMGVQMDLDFKLLETGSLFMAVLVTAFTLQDGTSHYLKGILLLLCYIVIGACFFVARQPAGHANSNGALLDVPTGSMSVQAA

SEQ ID NO: 7 - protein, 418 aa, Zea Mays
MHASGKKSDLALLRKVPCAPLRRLLDNLQEVLLATKLALLFPAVLLAIAARIFHFGQEWVFVLSLIGLVPLAERLSFLTEQVAFYIGPTVGGLLNATFGNVTEVIIAIFALYQGKVVVVKCSLLGSVLSNLLLVLGTSLFFGGLANLGTEQLYDKMQVDVNTGLLILGVLCHSLPLMLRYAVSSGEHAESSWDSGLELSRACSIVMLLAYVAYLFFQLKTHRQLFEPQPQEVEDDGDDSVSQDEAVLGFSSAMIWLGVMTLMTALLSEFVVSTIEAASESWELSVSFISVILIPIVGNAAEHAGAVIFAFKNNLDITLGVSLGSATQISMFVVPLSVLVAWIMGVPMDLDFNLLETGCLFLAILVTAFTLQDGSSHYLKGLLLVFCYIVISLCFFVLRQHGNGSNDDQVGVASKPWRI

(19) United States
(12) Patent Application Publication
(10) Pub. No.: US 2004/0229208 A1
(43) Pub. Date: Nov. 18, 2004

(54) Methods for the identification of inhibitors of CAX1-like Ca+2/H+ antiporter activity in plants

(76) Inventors: M. DeZwaan Todd, Apex, NC (US); Lining Guo, Chapel Hill, NC (US); Keith Davis, Durham, NC (US); Adel Zayed, Durham, NC (US); Susanne Kjemtrup, Chapel Hill, NC (US); Douglas Boyes, Chapel Hill, NC (US); Brenda Warrick, Cary, NC (US); Joseph Cameron Mitchell, Chapel Hill, NC (US); Robert Ascenzi, Cary, NC (US); Carol Hamilton, Apex, NC (US); Jeffrey Woessner, Hillsborough, NC (US); Jorn Gorlach, Manchester, NJ (US); Neil Hoffman, Chapel Hill, NC (US); Rao Mulpuri, Apex, NC (US)

Correspondence Address: Icoria, Inc., 108 T.W. ALEXANDER DRIVE, P O BOX 14528, RTP, NC, 27709-4528, US

(21) Appl. No.: 10/436,223

(22) Filed: May 12, 2003

(51) Int. Cl. C12Q 1/00

(52) U.S. Cl. 435/4

(57) ABSTRACT

The present inventors have discovered that CAX1-like H+/Ca⁺² antiporter is essential for plant growth. Specifically, the inhibition of CAX1-like H+/Ca⁺² antiporter gene expression in plant seedlings results in reduced growth and abnormal development. Thus, CAX1-like H+/Ca⁺² antiporter is useful as a target for the identification of herbicides. Accordingly, the present invention provides methods for the identification of herbicides by measuring the activity of a CAX1-like H+/Ca⁺² antiporter in the presence and absence of a compound, where an alteration of CAX1-like H+/Ca⁺² antiporter activity in the presence of the compound indicates the compound as a candidate for a herbicide.

### FIELD OF THE INVENTION

The invention relates generally to plant molecular biology. In particular, the invention relates to methods for the identification of herbicides.

### BACKGROUND OF THE INVENTION

The traditional approach to herbicide development is to spray chemicals, produced in milligram or greater quantity, on plants and then to monitor plant growth. While spray and observe approach has resulted in the identification of commercially important herbicides, rising costs, and efficacy and safety concerns are challenging its future productivity. Accordingly, there is a need to identify herbicide targets so that compound libraries can be screened for herbicidal activity in higher through-put in vitro or cell-based assays. Inhibitors of the identified targets can then be selected and confirmed as having herbicidal activity using conventional assays.

Calcium plays a central role in signal transduction by eukaryotic cells. A complex mechanism exists to control Ca⁺² in a localized fashion. Ca⁺² -ATPase, Ca⁺²/H+ antiporter (also referred to as Ca+²/H+ exchanger), Ca+²/Na+ antiporter, and Ca+² channel are involved in the regulation of cytosolic Ca⁺² concentration (Yuasa & Maeshima (2000) Plant Physiology 124: 1069-1078). In animals, Ca⁺² is primarily mobilized from the endoplasmic reticulum. In plants and fungi the vacuole is a primary pool of Ca⁺² and the vacuolar membrane has two distinct active transport systems for Ca⁺²: Ca⁺²-ATPase and Ca⁺²/H+ antiporter (Sanders et al. (1999) Plant Cell 11: 691-706; Sze et al. (2000) Annu. Rev. Plant Physiol. Plant Mol. Biol. 51: 433-462).

Yeast mutants defective in Ca⁺² transport have been characterized. Yeast vcx1 pmc1 mutants deleted for the vacuolar H+/Ca⁺² antiporter (VCX1) and vacuolar P-type Ca⁺²-ATPase (PMC1) genes are hypersensitive to Ca⁺² in the growth medium (Cunningham & Fink (1994) J. Cell Biol. 124: 351-363; Cunningham & Fink (1996) Mol. Cell Biol. 16: 2226-2237). Three Arabidopsis thaliana genes, CAX1, CAX2, and CAX3, have been shown to suppress the yeast vcx1 pmc1 mutant Ca⁺² accumulation defect (Hirschi et al. (1996) Proc. Natl. Acad. Sci. 93: 8782-8786; Shigaki T. et al. (2001) J. Biol. Chem. 276:43152-9). The Arabidopsis genes, CAX1 and CAX2, encode high efficiency and low efficiency H+/Ca⁺² antiporters, respectively. The CAX3 gene encodes a CAX1-like H+/Ca⁺² antiporter.

The present invention discloses CAX1-like H+/Ca⁺² antiporters as targets for the evaluation of plant growth regulators, especially herbicide compounds, in plants.

### SUMMARY OF THE INVENTION

The present inventors have discovered that antisense expression of a CAX1-like H+/Ca⁺² antiporter cDNA (Accession No.: AF256228) in Arabidopsis causes reduced growth and abnormal development. Thus, the present inventors have discovered that CAX1-like H+/Ca⁺² antiporter is essential for normal plant development and growth, and is useful as a target for the identification of herbicides.

Accordingly, in one embodiment the present invention provides methods for identifying a compound as a candidate for a herbicide, comprising: measuring the activity of a CAX1-like H+/Ca⁺² antiporter in the presence and absence of a compound, wherein an alteration of the CAX1-like H+/Ca⁺² antiporter activity in the presence of the compound indicates the compound as a candidate for a herbicide.

In another embodiment, the present invention provides methods for the identification of compounds that inhibit CAX1-like H+/Ca⁺² antiporter expression or activity, comprising: measuring the growth in a high Ca⁺² medium of a Ca⁺² sensitive pmc1 vcx1 strain of mutant yeast cells in the presence and absence of a compound, wherein the mutant yeast cells express a heterologous CAX1-like H+/Ca⁺² antiporter polypeptide that rectifies the Ca⁺² sensitive phenotype; measuring the growth in a normal Ca⁺² medium of a Ca⁺² sensitive pmc1 vcx1 strain of mutant yeast cells in the presence and absence of the compound, wherein the mutant yeast cells express a heterologous CAX1-like H+/Ca⁺² antiporter polypeptide that rectifies the Ca⁺² sensitive phenotype; and comparing the growth in steps (a) and (b), wherein a decrease in growth in step (a) in the presence relative to the absence of the compound, and no change in growth in step (b) between the presence and absence of the compound indicates the compound as a candidate for a herbicide.

In another embodiment, the invention provides methods for identifying a compound as a candidate for a herbicide, comprising: measuring the expression of a CAX1-like H+/Ca⁺² antiporter in a plant, or tissue thereof, in the presence and absence of a compound; and comparing the expression of the CAX1-like H+/Ca⁺² antiporter in the presence and absence of the compound, wherein an altered expression in the presence of the compound indicates that the compound is a candidate for a herbicide.

### DETAILED DESCRIPTION OF THE INVENTION

The term “bDNA” refers to branched DNA.

As used herein, the terms “CAX1-like H+/Ca⁺² antiporter,” “Ca⁺²/H+exchanger” and “Ca⁺²/H+ exchange protein” are interchangeable, and refer to an enzyme that catalyzes vacuolar H+/Ca⁺² antiporter activity. As used herein, the term “CAX1-like H+/Ca⁺² antiporter” means either a nucleic acid encoding a polypeptide or a polypeptide, wherein the polypeptide has at least 40%, 45%, 50%, 55%, 60%, 65%, 70%, 75%, 80%, 85%, 90%, 91%, 92%, 95%, 96%, 97%, 98%, or 99% sequence identity or each integer unit of sequence identity from 40-100% in ascending order to either Arabidopsis CAX1-like H+/Ca⁺² antiporter protein (CAX3; SEQ ID NO:2) or Arabidopsis truncated CAX1-like H+/Ca⁺² antiporter protein (SEQ ID NO:4) and at least 10%, 25%, 50%, 75%, 80%, 90%, 95%, or 99% activity or each integer unit of activity from 10-100% in ascending order of the activity of Arabidopsis CAX1-like H+/Ca⁺² antiporter protein (CAX3; SEQ ID NO:2) or Arabidopsis truncated CAX1-like H+/Ca⁺² antiporter protein (SEQ ID NO:4), respectively. Examples of CAX1-like H+/Ca⁺² antiporter's include, but are not limited to, CAX1-like H+/Ca⁺² antiporter from Vigna radiate, CAX1-like H+/Ca⁺² antiporter from Oryza sativa and CAX1-like H+/Ca⁺² antiporter from Zea Mays.

By “Ca⁺² sensitive” is meant that growth of a pmc1 vcx1 mutant is inhibited on culture medium containing a high concentration of Ca⁺² but not one containing a normal concentration of Ca⁺². By “high Ca⁺² medium” is meant a medium containing sufficient Ca⁺² to cause a reduction in growth of a pmc1 vcx1 mutant relative to a wild-type yeast strain. It is understood by one of ordinary skill in the art that the concentration of Ca⁺² in a high Ca⁺² medium will vary depending on the type and composition of growth medium used (e.g. liquid versus solid), the length of the growth period measured, and with the particular mutant yeast strain. Examples of normal concentrations of Ca⁺² for growth of a pmc1 vcx1 mutant yeast strain on solid media range from 0 to 100 mM. Examples of high concentrations of Ca⁺² for growth of a pmc1 vcx1 mutant yeast strain on solid media range from 200 to 300 mM. Normal concentrations of Ca⁺² for growth of a pmc1 vcx1 mutant yeast strain in liquid media are lower than those for growth on solid media. For example, a high concentration of Ca⁺² for growth of a pmc1 vcx1 mutant yeast strain in liquid media is 100 mM.

As used herein, the term “CDNA” means complementary deoxyribonucleic acid.

As used herein, the term “ELISA” means enzyme-linked immunosorbent assay.

As used herein, the term “GUS” means β-glucouronidase.

The term “herbicide,” as used herein, refers to a compound useful for killing or suppressing the growth of at least one plant, plant cell, plant tissue or seed.

The phrase “heterologous CAX1-like H+/Ca⁺² antiporter,” as used herein, refers to any CAX1-like H+/Ca⁺² antiporter polypeptide that is encoded by a nucleic acid molecule that has been transformed or introduced into mutant yeast cells being used in a cell based assay for identifying inhibitors of CAX1-like H+/Ca⁺² antiporter activity.

The term “inhibitor,” as used herein, refers to a chemical substance that inactivates the enzymatic activity of CAX1-like H+/Ca⁺² antiporter or substantially reduces the level of enzymatic activity, wherein “substantially” means a reduction at least as great as the standard deviation for a measurement, preferably a reduction by 50%, more preferably a reduction of at least one magnitude, i.e. to 10%. The inhibitor may function by interacting directly with the enzyme, a cofactor of the enzyme, the substrate of the enzyme, or any combination thereof.

A polynucleotide is “introduced” into a plant cell by any means, including transfection, transformation or transduction, electroporation, particle bombardment, agroinfection and the like. The introduced polynucleotide is maintained in the cell stably if it is incorporated into a non-chromosomal autonomous replicon or integrated into the plant chromosome. Alternatively, the introduced polynucleotide is present on an extra-chromosomal non-replicating vector and be transiently expressed or transiently active.

As used herein, the term “PCR” means polymerase chain reaction.

The “percent (%) sequence identity” between two polynucleotide or two polypeptide sequences is determined according to the either the BLAST program (Basic Local Alignment Search Tool, Altschul and Gish (1996) Meth Enzymol 266: 460-480; Altschul (1990) J Mol Biol 215: 403 -410) or using Smith Waterman Alignment (Smith and Waterman (1981) Adv Appl Math 2:482) using the default settings and the version current at the time of filing). It is understood that for the purposes of determining sequence identity when comparing a DNA sequence to an RNA sequence, a thymine nucleotide is equivalent to a uracil nucleotide.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

By “plant CAX1-like H+/Ca⁺² antiporter” is meant a protein found in at least one plant, and which catalyzes vacuolar H+/Ca⁺² antiporter activity. The CAX1-like H+/Ca⁺² antiporter is from any plant, including monocots, dicots, C3 plants, C4 plants and/or plants that are classified as neither C3 nor C4 plants.

By “polypeptide” is meant a chain of at least four amino acids joined by peptide bonds. The chain is linear, branched, circular or combinations thereof. The polypeptides may contain amino acid analogs and other modifications, including, but not limited to glycosylated or phosphorylated residues.

The present inventors have discovered that inhibition of CAX1-like H+/Ca⁺² antiporter gene expression inhibits the growth and development of plant seedlings. Antisense expression of Arabidopsis thaliana CAX3 cDNA (AF256228; SEQ ID NO:1) resulted in abnormal growth and development. The protein encoded by the Arabidopsis CAX3 gene (SEQ ID NO:2) has a high degree of homology to Arabidopsis CAX1 high affinity H+/Ca⁺² antiporter (AAB05913). Thus, the inventors are the first to demonstrate that CAX1-like H+/Ca⁺² antiporters are useful targets for the identification of herbicides.

Accordingly, the invention provides methods for identifying compounds that inhibit CAX1-like H+/Ca⁺² antiporter protein activity. Such methods include binding assays, activity assays and assays for CAX1-like H+/Ca⁺² antiporter gene expression. The compounds identified by the methods of the invention are useful as herbicides.

In one embodiment, the invention provides a method for the identification of a compound as a herbicide, comprising: measuring the activity of a CAX1-like H+/Ca⁺² antiporter in the presence and absence of the compound, wherein an alteration of the CAX1-like H+/Ca⁺² antiporter activity in the presence of the compound indicates the compound as a candidate for a herbicide.

By “CAX1-like H+/Ca⁺² antiporter” is meant an enzyme that catalyzes vacuolar H+/Ca⁺² antiporter activity. In one embodiment of the invention, the CAX1-like H+/Ca⁺² antiporter has the amino acid sequence of a naturally occurring CAX1-like H+/Ca⁺² antiporter found in a plant, animal or microorganism. In another embodiment of the invention, the CAX1-like H+/Ca⁺² antiporter has an amino acid sequence derived from a naturally occurring sequence. In another embodiment the CAX1-like H+/Ca⁺² antiporter is a plant CAX1-like H+/Ca⁺² antiporter.

One example of a cDNA encoding anArabidopsis CAX1-like H+/Ca⁺² antiporter is set forth in SEQ ID NO:1 (TIGR database locus At3g51860; CAX3). The CAX1-like H+/Ca⁺² antiporter polypeptide encoded by SEQ ID NO:1 is set forth in SEQ ID NO:2. A nucleic acid molecule encoding an N-terminal 55 amino acid truncated and C-terminal 6-His tag peptide fusion of Arabidopsis CAX1-like H+/Ca⁺² antiporter is set forth in SEQ ID NO:3. The fusion polypeptide encoded by SEQ ID NO:3 is set forth in SEQ ID NO:4. Other examples of CAX1-like H+/Ca⁺² antiporters include the Vigna radiata CAX1-like H+/Ca⁺² antiporter protein set forth in SEQ ID NO:5 (Accession No. BAA25753). Another example of a CAX1-like H+/Ca⁺² antiporter is a Oryza sativa CAX1-like H+/Ca⁺² antiporter protein set forth in SEQ ID NO:6 (Accession No. BAB89095). Another example of a CAX1-like H+/Ca⁺² antiporter is a Zea Mays CAX1-like H+/Ca⁺² antiporter protein set forth in SEQ ID NO:7 (Accession No. AAF91350). Vigna radiate, Oryza sativa and Zea Mays CAX1-like H+/Ca⁺² antiporters that have N-terminal amino acid truncations similar to that of SEQ ID NO:4 are also useful in the methods of the invention.

In one embodiment, the CAX1-like H+/Ca⁺² antiporter is an Arabidopsis CAX1-like H+/Ca⁺² antiporter. Arabidopsis species include, but are not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii.

In various embodiments, the CAX1-like H+/Ca⁺² antiporter can be from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

CAX1-like H+/Ca⁺² antiporter polypeptides having at least 40% sequence identity with either Arabidopsis CAX1-like H+/Ca⁺² antiporter protein (CAX3; SEQ ID NO:2) or Arabidopsis truncated CAX1-like H+/Ca⁺² antiporter protein (SEQ ID NO:4) protein are also useful in the methods of the invention. In one embodiment, the sequence identity is at least 40%, 45%, 50%, 55%, 60%, 65%, 70%, 75%, 80%, 85%, 90%, 91%, 92%, 93%, 94%, 95%, 99%, or any integer from 40-100% sequence identity in ascending order with either Arabidopsis CAX1-like H+/Ca⁺² antiporter protein (CAX3; SEQ ID NO:2) or Arabidopsis truncated CAX1-like H+/Ca⁺² antiporter protein (SEQ ID NO:4). In addition, it is preferred that CAX1-like H+/Ca⁺² antiporter polypeptides of the invention have at least 10% of the activity of either Arabidopsis CAX1-like H+/Ca⁺² antiporter protein (CAX3; SEQ ID NO:2) or Arabidopsis truncated CAX1-like H+/Ca⁺² antiporter protein (SEQ ID NO:4). CAX1-like H+/Ca 2 antiporter polypeptides of the invention have at least 10%, 15%, 20%, 25%, 30%, 35%, 40%, 45%, 50%, 55%, 60%, 65%, 70%, 75%, 80%, 85% or at least 90% of the activity of Arabidopsis CAX1-like H+/Ca⁺² antiporter protein (SEQ ID NO:2) or Arabidopsis truncated CAX1-like H+/Ca⁺² antiporter protein (SEQ ID NO:4).

Polypeptides consisting essentially of SEQ ID NO:4 are also useful in the methods of the invention. For the purposes of the present invention, a polypeptide consisting essentially of SEQ ID NO:4 has at least 90% sequence identity with Arabidopsis CAX1-like H+/Ca⁺² antiporter fusion protein (SEQ ID NO:4) and at least 10% of the activity of SEQ ID NO:4. A polypeptide consisting essentially of SEQ ID NO:4 has at least 90%, 91%, 92%, 93%, 94%, 95%, 96%, 97% 98%, or 99% sequence identity with SEQ ID NO:4 and at least 25%, 50%, 75%, or 90% of the activity of Arabidopsis CAX1-like H+/Ca⁺² antiporter (SEQ ID NO:4).

Examples of polypeptides consisting essentially of SEQ ID NO:4 include, but are not limited to, polypeptides having the amino acid sequence of SEQ ID NO:4 with the exception that one or more of the amino acids are substituted with structurally similar amino acids providing a “conservative amino acid substitution.” Conservative amino acid substitutions are well known to those of skill in the art. Particular examples of polypeptides consisting essentially of SEQ ID NO:4 include polypeptides having 1, 2, or 3 conservative amino acid substitutions relative to SEQ ID NO:4.

Other examples of polypeptides consisting essentially of SEQ ID NO:4 include polypeptides having the sequence of SEQ ID NO:4, but with truncations at either or both the 3′ and the 5′ end. For example, polypeptides consisting essentially of SEQ ID NO:4 include polypeptides having 1, 2, or 3 amino acids residues removed from either or both 3′ and 5′ ends relative to SEQ ID NO:4. Additional examples of polypeptides consisting essentially of SEQ ID NO:4 include polypeptides having 1, 2, 3, 4, 5, 6, 7, 8, 9, or 10 fewer amino acids residues truncated from the N-terminus relative to SEQ ID NO:4. In addition, CAX1-like H+/Ca⁺² antiporter polypeptides consisting essentially of SEQ ID NO:4 can be fusion proteins, such as SEQ ID NO:4 but having other fused polypeptides or amino acid sequences to aid in secretion and/or isolation as is known to those of skill in the art.

Fragments of a CAX1-like H+/Ca⁺² antiporter polypeptide are useful in the methods of the invention. In one embodiment, the CAX1-like H+/Ca⁺² antiporter fragments include an intact or nearly intact epitope that occurs on the biologically active wild-type CAX1-like H+/Ca⁺² antiporter. For example, the fragments comprise at least 10 consecutive amino acids of CAX1-like H+/Ca⁺² antiporter of SEQ ID NO:2. The fragments comprise at least 15, 20, 25, 30, 35, 40, 50, 60, 70, 80, 90, 100, 125, 150, 175, 200, 225, 250, 275, 300, 325, 350, 375, 400, 425 or at least 458 consecutive amino acid residues of CAX1-like H+/Ca⁺² antiporter of SEQ ID NO:2. Polypeptides comprising at least 50 amino acids having at least 50%, 60%, 70%, 80%, 90%, 95%, 96%, 97%, 98% or 99% sequence identity with at least 50 consecutive amino acid residues of SEQ ID NO:2 are also useful in the methods of the invention. In one embodiment, the fragment is from an Arabidopsis CAX1-like H+/Ca⁺² antiporter. In one embodiment, the fragment contains an amino acid sequence conserved among plant CAX1-like H+/Ca⁺² antiporter sequences.

Thus, in another embodiment, the invention provides a method for identifying a compound as a candidate for a herbicide, comprising: contacting a compound with a CAX1-like H+/Ca⁺² antiporter polypeptide selected from the group consisting of: a polypeptide set forth in SEQ ID NO:2 or SEQ ID NO:4; a CAX1-like H+/Ca⁺² antiporter polypeptide consisting essentially of SEQ ID NO:4; a polypeptide comprising at least 10 consecutive amino acids of SEQ ID NO:2; a CAX1-like H+/Ca⁺² antiporter polypeptide having at least 50% sequence identity with SEQ ID NO:2 or SEQ ID NO:4; and a CAX1-like H+/Ca⁺² antiporter polypeptide comprising at least 50 amino acids having at least 50% sequence identity with at least 50 consecutive amino acid residues of SEQ ID NO:2 or SEQ ID NO:4; and detecting the presence and/or absence of binding between the compound and the polypeptide, wherein binding indicates that the compound is a candidate for a herbicide.

Any technique for detecting the binding of a ligand to its target is useful in the methods of the invention. For example, the ligand and target are combined in a buffer. Many methods for detecting the binding of a ligand to its target are known in the art, and include, but are not limited to the detection of an immobilized ligand-target complex or the detection of a change in the properties of a target when it is bound to a ligand. For example, in one embodiment, an array of immobilized candidate ligands is provided. The immobilized ligands are contacted with a CAX1-like H+/Ca⁺² antiporter protein or a fragment or variant thereof, the unbound protein is removed and the bound CAX1-like H+/Ca⁺² antiporter is detected. In a preferred embodiment, bound CAX1-like H+/Ca⁺² antiporter is detected using a labeled binding partner, such as a labeled antibody. In a variation of this assay, CAX1-like H+/Ca ⁺² antiporter is labeled prior to contacting the immobilized candidate ligands. Preferred labels include fluorescent or radioactive moieties. In other embodiments of the invention, detection methods include fluorescence correlation spectroscopy (FCS) and FCS-related confocal nanofluorimetric methods.

In another embodiment of the invention, compounds are tested as candidate herbicides based on ability to inhibit CAX1-like H+/Ca⁺² antiporter enzyme activity. The compounds are tested using either in vitro or cell based enzyme assays. Alternatively, compounds are tested by direct application to a plant or plant cell, or expressing it therein, and monitoring the plant or plant cell for changes or decreases in growth, development, viability or alterations in gene expression.

A decrease in growth occurs where the herbicide candidate causes at least a 10% decrease in the growth of the plant or plant cells, as compared to the growth of the plants or plant cells in the absence of the herbicide candidate. A decrease in viability occurs where at least 20% of the plants cells, or portions of the plant contacted with the herbicide candidate, are nonviable. Preferably, the growth or viability will be decreased by at least 40%. More preferably, the growth or viability will be decreased by at least 50%, 75%, or at least 90% or more. Methods for measuring plant growth and cell viability are known to those skilled in the art. It is possible that a candidate compound may have herbicidal activity only for certain plants or certain plant species.

The ability of a compound to inhibit CAX1-like H+/Ca⁺² antiporter activity can be detected using cell based assays in which a CAX1-like H+/Ca⁺² antiporter polypeptide complements the Ca⁺² sensitivity of a yeast pmc1 vcx1 double mutant. Growth of the pmc1 vcx1 mutant is inhibited on culture medium containing a high concentration of Ca⁺². Expression of a heterologous CAX1-like H+/Ca⁺² antiporter protein relieves the inhibition and enables the mutant to grow in the presence of a high Ca⁺² concentration. CAX1-like H+/Ca⁺² antiporter inhibitors are identified by their ability to confer Ca⁺² sensitive growth on the CAX1-like H+/Ca⁺² antiporter-expressing pmc1 vcx1 mutant.

Thus, in one embodiment, the invention provides methods for the identification of compounds that inhibit CAX1-like H+/Ca⁺² antiporter activity, comprising: measuring the growth in a high Ca⁺² medium of a Ca⁺² sensitive pmc1 vcx1 strain of mutant yeast cells in the presence and absence of a compound, wherein the mutant yeast cells express a heterologous CAX1-like H+/Ca⁺² antiporter polypeptide that rectifies the Ca⁺² sensitive phenotype; measuring the growth in a normal Ca⁺² medium of a Ca⁺² sensitive pmc1 vcx1 strain of mutant yeast cells in the presence and absence of the compound, wherein the mutant yeast cells express a heterologous CAX1-like H+/Ca ⁺² antiporter polypeptide that rectifies the Ca⁺² sensitive phenotype; and comparing the growth in steps (a) and (b), wherein a decrease in growth in step (a) in the presence relative to the absence of the compound, and no change in growth in step (b) between the presence and absence of the compound indicates the compound as a candidate for a herbicide. The phrase “heterologous CAX1-like H+/Ca⁺² antiporter” is herein intended to mean any CAX1-like H+/Ca⁺² antiporter polypeptide that is encoded by a nucleic acid molecule that has been transformed or introduced into the mutant yeast cells.

In one embodiment of the invention, the CAX1-like H+/Ca⁺² antiporter is the polypeptide set forth in SEQ ID NO:2. In another embodiment, the CAX1-like H+/Ca⁺² antiporter is the polypeptide set forth in SEQ ID NO:4. In another embodiment, the CAX1-like H+/Ca⁺² antiporter is a polypeptide consisting essentially of SEQ ID NO:4. In another embodiment, the CAX1-like H+/Ca⁺² antiporter is an Arabidopsis CAX1-like H+/Ca⁺² antiporter polypeptide. In another embodiment, the CAX1-like H+/Ca⁺² antiporter is a plant CAX1-like H+/Ca⁺² antiporter. In another embodiment the CAX1-like H+/Ca⁺² antiporter is Vigna radiata CAX 1-like H+/Ca⁺² antiporter set forth in SEQ ID NO:5. In another embodiment the CAX1-like H+/Ca⁺² antiporter is Oryza sativa CAX1-like H+/Ca⁺² antiporter set forth in SEQ ID NO:6. In another embodiment the CAX1-like H+/Ca⁺² antiporter is Zea Mays CAX1-like H+/Ca⁺² antiporter set forth in SEQ ID NO:7.

Enzymatically active fragments of Arabidopsis CAX1-like H+/Ca⁺² antiporter set forth in SEQ ID NO:2 or SEQ ID NO:4 are also useful in the methods of the invention. For example, an enzymatically active polypeptide comprising at least 50 consecutive amino acid residues and at least 10% of the activity of Arabidopsis CAX1-like H+/Ca⁺² antiporter set forth in SEQ ID NO:2 or SEQ ID NO:4 are useful in the methods of the invention. The fragments comprise at least 15, 20, 25, 30, 35, 40, 50, 60, 70, 80, 90, 100, 125, 150, 175, 200, 225, 250, 275, 300, 325, 350, 375, 400, 425 or at least 458 consecutive amino acid residues of CAX1-like H+/Ca⁺² antiporter of SEQ ID NO:2 or SEQ ID NO:4. In addition, enzymatically active fragments of CAX1-like H+/Ca⁺² antiporter's useful in the methods of the invention include polypeptides comprising at least 50 amino acids having at least 10% of the activity of SEQ ID NO:2 or SEQ ID NO:4 and at least 50%, 60%, 70%, 80%, 90%, 95%, 96%, 97%, 98% or 99% sequence identity with at 50 consecutive amino acid residues of SEQ ID NO:2 or SEQ ID NO:4, respectively, are useful in the methods of the invention. Most preferably, the enzymatically active polypeptide comprises at least 50 amino acids, has at least 50% sequence identity with at least 50 consecutive amino acid residues of SEQ ID NO:2 or SEQ ID NO:4 and at least 25%, 75% or at least 90% of the activity thereof.

Thus, in other embodiments of the invention, the CAX1-like H+/Ca⁺² antiporter is a polypeptide selected from the group consisting of: a polypeptide having at least 50% sequence identity with Arabidopsis CAX1-like H+/Ca⁺² antiporter set forth in SEQ ID NO:2 or SEQ ID NO:4 and having at least 10% of the activity thereof; a polypeptide comprising at least 50 consecutive amino acids ofArabidopsis CAX1-like H+/Ca⁺² antiporter set forth in SEQ ID NO:2 or SEQ ID NO:4 and having at least 10% of the activity thereof; and a polypeptide comprising at least 50 amino acids, having at least 50% sequence identity with at least 50 consecutive amino acids of Arabidopsis CAX1-like H+/Ca⁺² antiporter set forth in SEQ ID NO:2 or SEQ ID NO:4 and having at least 10% of the activity thereof.

As an alternative to cell-based assays, the invention also provides plant based assays. In one embodiment, the invention provides a method for identifying a compound as a candidate for a herbicide, comprising: a) measuring the expression or activity of a CAX1-like H+/Ca⁺² antiporter in a plant, or tissue thereof, in the absence of a compound; b) measuring the expression or activity of the CAX1-like H+/Ca⁺² antiporter in the plant, or tissue thereof, in the presence of the compound; and c) comparing the expression or activity of the CAX1-like H+/Ca⁺² antiporter in steps (a) and (b), wherein an altered expression or activity in the presence of the compound indicates that the compound is a candidate for a herbicide. In one embodiment, the plant or tissue thereof is Arabidopsis thaliana.

In the methods of the invention, expression of a CAX1-like H+/Ca⁺² antiporter in a plant, or tissue thereof, is measured by detecting the CAX1-like H+/Ca⁺² antiporter primary transcript or mRNA, CAX1-like H+/Ca⁺² antiporter polypeptide or CAX1-like H+/Ca⁺² antiporter enzymatic activity. Methods for detecting the expression of RNA and proteins are known to those skilled in the art. (See, for example, Current Protocols in Molecular Biology, Ausubel et al., eds., Greene Publishing and Wiley-Interscience, New York, 1995). However, the method of detection is not critical to the invention. Methods for detecting CAX1-like H+/Ca⁺² antiporter RNA include, but are not limited to, amplification assays such as quantitative PCR, and/or hybridization assays such as Northern analysis, dot blots, slot blots, in-situ hybridization, transcriptional fusions using a CAX1-like H+/Ca⁺² antiporter promoter fused to a reporter gene, bDNA assays, and microarray assays.

Methods for detecting protein expression include, but are not limited to, immunodetection methods such as Western blots, His Tag and ELISA assays, polyacrylamide gel electrophoresis, mass spectroscopy, and enzymatic assays. Also, any reporter gene system is useful to detect CAX1-like H+/Ca⁺² antiporter protein expression. For detection using gene reporter systems, a polynucleotide encoding a reporter protein is fused in frame with CAX1-like H+/Ca⁺² antiporter, so as to produce a chimeric polypeptide. Methods for using reporter systems are known to those skilled in the art. Examples of reporter genes include, but are not limited to, chloramphenicol acetyltransferase (Gorman et al. (1982) Mol Cell Biol 2: 1104; Prost et al. (1986) Gene 45: 107-111), β-galactosidase (Nolan et al. (1988) Proc Natl Acad Sci USA 85: 2603-2607), alkaline phosphatase (Berger et al. (1988) Gene 66: 10), luciferase (De Wet et al. (1987) Mol Cell Biol 7: 725-737), β-glucuronidase (GUS), fluorescent proteins, chromogenic proteins and the like.

Chemicals, compounds, or compositions identified by the above methods as modulators of CAX1-like H+/Ca⁺² antiporter expression or activity are useful for controlling plant growth. For example, compounds that inhibit plant growth are applied to a plant or expressed in a plant to prevent plant growth. Thus, the invention provides a method for inhibiting plant growth, comprising contacting a plant with a compound identified by the methods of the invention as having herbicidal activity.

Herbicides and herbicide candidates identified by the methods of the invention are useful for controlling the growth of undesired plants, including including monocots, dicots, C3 plants, C4 plants, and plants that are neither C3 nor C4 plants. Examples of undesired plants include, but are not limited, to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

### EXPERIMENTAL

Plant Growth Conditions

Unless, otherwise indicated, all plants were grown in Scotts Metro-Mix™ soil (the Scotts Company) or a similar soil mixture in an environmental growth room at 22° C., 65% humidity, 65% humidity and a light intensity of ˜100 μ-E m⁻² s⁻¹ supplied over 16 hour day period.

Seed Sterilization

All seeds were surface sterilized before sowing onto phytagel plates using the following protocol.

1. Place approximately 20-30 seeds into a labeled 1.5 ml conical screw cap tube. Perform all remaining steps in a sterile hood using sterile technique.

2. Fill each tube with 1 ml 70% ethanol and place on rotisserie for 5 minutes.

3. Carefully remove ethanol from each tube using a sterile plastic dropper; avoid removing any seeds.

4. Fill each tube with 1 ml of 30% Clorox and 0.5% SDS solution and place on rotisserie for 10 minutes.

5. Carefully remove bleach/SDS solution.

6. Fill each tube with 1 ml sterile DI H₂O; seeds should be stirred up by pipetting of water into tube. Carefully remove water. Repeat 3 to 5 times to ensure removal of Clorox/SDS solution.

7. Fill each tube with enough sterile dI H₂O for seed plating (˜200-400 μl). Cap tube until ready to begin seed plating.

Plate Growth Assays

Surface sterilized seeds were sown onto plate containing 40 ml half strength sterile MS (Murashige and Skoog, no sucrose) medium and 1% Phytagel using the following protocol:

1. Using pipette man and 200 μl tip, carefully fill tip with seed solution. Place 10 seeds across the top of the plate, about 1/4 inch down from the top edge of the plate.

2. Place plate lid ¾ of the way over the plate and allow to dry for 10 minutes.

3. Using sterile micropore tape, seal the edge of the plate where the top and bottom meet.

4. Place plates stored in a vertical rack in the dark at 4° C. for three days.

5. Three days after sowing, the plates transferred into a growth chamber with a day and night temperature of 22 and 20° C., respectively, 65% humidity and a light intensity of ˜100 μ-E m⁻² s⁻¹ supplied over 16 hour day period.

6. Beginning on day 3, daily measurements are carried out to track the seedlings development until day 14. Seedlings are harvested on day 14 (or when root length reaches 6 cm) for root and rosette analysis.

### EXAMPLE 1

### Construction of a Transgenic Plant expressing the Driver

The “Driver” is an artificial transcription factor comprising a chimera of the DNA-binding domain of the yeast GAL4 protein (amino acid residues 1-147) fused to two tandem activation domains of herpes simplex virus protein VP16 (amino acid residues 413-490). Schwechheimer et al. (1998) Plant Mol Biol 36:195-204. This chimeric driver is a transcriptional activator specific for promoters having GAL4 binding sites. Expression of the driver is controlled by two tandem copies of the constitutive CaMV 35S promoter.

The driver expression cassette was introduced into Arabidopsis thaliana by agroinfection. Transgenic plants that stably expressed the driver transcription factor were obtained.

### EXAMPLE 2

### Construction of CAX1-Like H+/Ca⁺² Antiporter Antisense Expression Cassettes in a Binary Vector

A fragment of the Arabidopsis thaliana cDNA corresponding to SEQ ID NO:1 was ligated into the PacI/AscI sites of an E. coli/Agrobacterium binary vector in the antisense orientation to yield an antisense expression cassette and a constitutive chemical resistance expression cassette located between right and left T-DNA borders. In this construct, transcription of the antisense RNA is controlled by an artificial promoter active only in the presence of the driver transcription factor described above. The artificial promoter contains four contiguous binding sites for the GAL4 transcriptional activator upstream of a minimal promoter comprising a TATA box. The ligated DNA was transformed into E. coli. Kanamycin resistant clones were selected and purified. DNA was isolated from each clone and characterized by PCR and sequence analysis confirming the presence of the antisense expression cassette.

### EXAMPLE 3

### Transformation of Agrobacterium with the CAX1-Like H+/Ca⁺² Antiporter Antisense Expression Cassette

The binary vector described in Example 2 was transformed into Agrobacterium tumefaciens by electroporation. Transformed Agrobacterium colonies were isolated using chemical selection. DNA was prepared from purified resistant colonies and the inserts were amplified by PCR and sequenced to confirm sequence and orientation.

### EXAMPLE 4

### Construction of Arabidopsis CAX1-Like H+/Ca⁺² Antiporter Antisense Target Plants

The CAX1-like H+/Ca⁺² antiporter antisense expression cassette was introduced into Arabidopsis thaliana wild-type plants by the following method. Five days prior to agroinfection, the primary inflorescence of Arabidopsis thaliana plants grown in 2.5 inch pots were clipped to enhance the emergence of secondary bolts.

At two days prior to agroinfection, 5 ml LB broth (10 g/L Peptone, 5 g/L Yeast extract, 5 g/L NaCl, pH 7.0 plus 25 mg/L kanamycin added prior to use) was inoculated with a clonal glycerol stock of Agrobacterium carrying the desired DNA. The cultures were incubated overnight at 28° C. at 250 rpm until the cells reached stationary phase. The following morning, 200 ml LB in a 500 ml flask was inoculated with 500 μl of the overnight culture and the cells were grown to stationary phase by overnight incubation at 28° C. at 250 rpm. The cells were pelleted by centrifugation at 8000 rpm for 5 minutes. The supernatant was removed and excess media was removed by setting the centrifuge bottles upside down on a paper towel for several minutes. The cells were then resuspended in 500 ml infiltration medium (autoclaved 5% sucrose) and 250 μl/L Silwet L-77™ (84% polyalkyleneoxide modified heptamethyltrisiloxane and 16% allyloxypolyethyleneglycol methyl ether), and transferred to a one liter beaker.

The previously clipped Arabidopsis plants were dipped into the Agrobacterium suspension so that all above ground parts were immersed and agitated gently for 10 seconds. The dipped plants were then covered with a tall clear plastic dome to maintain the humidity, and returned to the growth room. The following day, the dome was removed and the plants were grown under normal light conditions until mature seeds were produced. Mature seeds were collected and stored desiccated at 4° C.

Transgenic Arabidopsis T1 seedlings were selected. Approximately 70 mg seeds from an agrotransformed plant were mixed approximately 4:1 with sand and placed in a 2 ml screw cap cryo vial. One vial of seeds was then sown in a cell of an 8 cell flat. The flat was covered with a dome, stored at 4° C. for 3 days, and then transferred to a growth room. The domes were removed when the seedlings first emerged. After the emergence of the first primary leaves, the flat was sprayed uniformly with a herbicide corresponding to the chemical resistance marker plus 0.005% Silwet (50 μl/L) until the leaves were completely wetted. The spraying was repeated for the following two days.

Ten days after the first spraying resistant plants were transplanted to 2.5 inch round pots containing moistened sterile potting soil. The transplants were then sprayed with herbicide and returned to the growth room. The herbicide resistant plants represented stably transformed T1 plants.

### EXAMPLE 5

### Effect of CAX1-Like H+/Ca⁺² Antiporter Antisense Expression in Arabidopsis Seedlings

The T1 CAX1-like H+/Ca⁺² antiporter antisense target plants from the transformed plant lines obtained in Example 4 were crossed with the Arabidopsis transgenic driver line described above. The resulting F1 seeds were then subjected to a plate assay to observe seedling growth over a 2-week period. Seedlings were inspected for growth and development. Antisense expression of the CAX1-like H+/Ca⁺² antiporter gene in three separate lines resulted in significantly impaired growth and abnormal development, indicating that the CAX1-like H+/Ca⁺² antiporter gene is an essential gene for normal plant growth and development. Six of eight plants from the first transgenic line, three of six plants from the second transgenic line, and five of seven plants from the third transgenic line showed reduced growth and/or abnormal development. Thus, each of the three transgenic lines containing the antisense construct for CAX1-like Ca⁺²/H+ antiporter exhibited significant seedling abnormalities.

### EXAMPLE 6

### Construction of a Ca⁺² Sensitive pmc1 vcx1 Mutant Yeast Strain

Saccharomyces cerevisiae having a double mutation in both PMC1 and VCX1 genes (pmc1 vcx1 double mutants) exhibits Ca⁺² Sensitivity (Cunningham & Fink (1994) J. Cell Biol. 124: 351-363; Cunningham & Fink (1996) Mol. Cell Biol. 16: 2226-2237). PMC1 encodes a vacuolar Ca⁺²-transporting ATPase. PMC1 is a member of the cation transporting P-type ATPase superfamily and functions to pump Ca⁺² out of the cytoplasm into the vacuole. VCX1 is a calcium transport (H+/Ca⁺² exchange) protein of the vacuolar membrane. VCX1 transports H+ out of the vacuole into the cytoplasm and Ca⁺² out of the cytoplasm into the vacuole. By “Ca⁺² sensitive” is meant that growth of a pmc1 vcx1 mutant is inhibited on culture medium containing a high concentration of Ca⁺² but not one containing a normal concentration of Ca^{+2.}

A pmc1Δ::KanMX mutant strain was purchased from Open Biosystems (cat. no. YSC1021-548753) and designated as PGY1. The entire PMC1 gene-coding region of strain PGY1 was replaced with a KanMX selectable marker, conferring resistance to the antibiotic geneticin (G418). In addition, PGY1 is a histidine auxotroph as a result of a his3Δ1 mutation. A vcx1 mutation was introduced into PGY1 using homologous recombination to replace the entire VCX1 coding region with the wild type HIS3 gene. Putative pmc1Δ::KanMX vcx1Δ::HIS3 double mutants were selected by their ability to grow on medium lacking histidine. The presence of both the pmc1 and vcx1 mutations was verified by PCR for two separate strains.

### EXAMPLE 7

### Complementation of the Ca⁺² Sensitive pmc1 vcx1 Mutant Yeast Strain by Expression of a Heterologous CAX1-Like H+/Ca⁺² Antiporter Protein

It has been demonstrated that the Ca⁺² sensitivity of yeast pmc1 vcx1 mutants can be complemented by Arabidopsis calcium exchangers CAX1, CAX2 and CAX3 (Hirsehi K. et al. (1996) Proc. Nat. Acad. Sci. 93:8782-6; Shigaki T. et al. (2001) J. Biol. Chem. 276:43152-9) and by the Toxoplasma gondii Ca⁺²-transporting ATPase TgA1 (Luo S. et al. (2001) EMBO J 20:55-64). Expression of CAX protein relieves the Ca⁺² inhibition and enables the mutant to grow in the presence of a high Ca⁺² concentration. The two pmc1Δ vcx1Δ strains from Example 6 were used to test for complementation by a heterologous CAX1-like H+/Ca⁺² antiporter gene.

The two PCR-verified mutant strains were transformed with yeast expression vectors expressing full-length (SEQ ID NO:1) and truncated forms (SEQ ID NO:3) of a CAX1-like H+/Ca⁺² antiporter to test whether the CAX1-like H+/Ca⁺² antiporter genes complemented the Ca⁺² -sensitivity of the pmc1 vcx1 mutants. As a control, the double mutant was transformed with empty vector. The Ca⁺² sensitivity of the strains was examined by spotting serial dilutions onto Ca⁺² containing raffinose-galactose plates that support maximal induction of the CAX1-like H+/Ca⁺² antiporter genes. Under these conditions all of the yeast strains grew equivalently at Ca⁺² concentrations between 0 and 100 mM. At Ca⁺² concentrations between 200 and 300 mM, pmc1 vcx1 mutants that expressed the truncated CAX1-like H+/Ca⁺² antiporter protein (SEQ ID NO:4) demonstrated more robust growth than the non-expressing mutants or the mutants expressing the full-length CAX1-like H+/Ca⁺² antiporter protein (SEQ ID NO:2).

The results are consistent with previous studies showing that the N-terminus of the Arabidopsis calcium exchangers contain an autoregulatory region that must be removed for the proteins to become functional in yeast (Luo S. et al. (2001) EMBO J 20:55-64). For further analysis, the vector control strain was designated, PGY15, and the truncated CAX1-like H+/Ca⁺² antiporter protein (SEQ ID NO:4)-expressing strain was designated, PGY17. RT-PCR was used to demonstrate that CAX1-like H+/Ca⁺² antiporter expression occurred in yeast strain PGY17 only upon induction with galactose. For this experiment, the control strain, PGY15, and the CAX1-like H+/Ca⁺² antiporter-expressing strain, PGY17, were cultured in growth media containing either glucose or raffinose and galactose. Glucose is the favored carbon source for yeast. When yeast is grown on glucose-containing media the expression of genes that are required for galactose metabolism is repressed. When yeast is grown on galactose or raffinose-galactose-containing media that lacks glucose, the galactose metabolic network is induced.

In strain PGY17, CAX1-like H+/Ca⁺² antiporter is under control of the GAL1-10 promoter and, thus, CAX1-like H+/Ca⁺² antiporter expression should only be observed when this strain is grown on galactose-containing medium. This is consistent with the observed RT-PCR results. CAX1-like H+/Ca⁺² antiporter expression was only observed in PGY17 grown in galactose-containing medium but not glucose-containing medium. As expected, no CAX1-like H+/Ca⁺² antiporter expression was observed in the control strain, PGY15, grown on either glucose or galactose-containing medium.

### EXAMPLE 8

### Assay for the Identification of Inhibitors of CAX1-Like H+/Ca⁺² Antiporter Activity

CAX1-like H+/Ca⁺² antiporter inhibitors are identified by the disappearance of CAX1-like H+/Ca⁺² antiporter complementation of growth of a CAX1-like H+/Ca⁺² antiporter-expressing pmc1 vcx1 mutant yeast strain grown on high Ca⁺² media. The identified inhibitors have no effect on growth of the CAX1-like H+/Ca⁺² antiporter-expressing pmc1 vcx1 mutant yeast strain grown on normal Ca⁺² media.

While the foregoing describes certain embodiments of the invention, it will be understood by those skilled in the art that variations and modifications may still fall within the scope of the invention.

What is claimed is:

1. A method for identifying a compound as a candidate for a herbicide, comprising:
a) measuring the growth in a high Ca⁺² medium of a Ca⁺² sensitive pmc1 vex1 strain of mutant yeast cells in the presence and absence of a compound, wherein the mutant yeast cells express a heterologous CAX1-like H+/Ca⁺² antiporter polypeptide that rectifies the Ca⁺² sensitive phenotype;
b) measuring the growth in a normal Ca⁺² medium of a Ca⁺² sensitive pmc1 vex1 strain of mutant yeast cells in the presence and absence of the compound, wherein the mutant yeast cells express a heterologous CAX1-like H+/Ca⁺² antiporter polypeptide that rectifies the Ca⁺² sensitive phenotype; and
c) comparing the growth in steps (a) and (b), wherein a decrease in growth in step (a) in the presence relative to the absence of the compound, and no change in growth in step (b) between the presence and absence of the compound indicates the compound as a candidate for a herbicide.

2. The method of claim 1, wherein the CAX1-like H+/Ca⁺² antiporter polypeptide is a plant CAX1-like H+/Ca⁺² antiporter polypeptide.

3. The method of claim 2, wherein the plant is a dicot.

4. The method of claim 2, wherein the plant is a monocot.

5. The method of claim 2, wherein the plant is other than a C3 plant.

6. The method of claim 2, wherein the plant is other than a C4 plant.

7. The method of claim 1, wherein the CAX1-like H+/Ca⁺² antiporter polypeptide is an Arabidopsis CAX1-like H+/Ca⁺² antiporter polypeptide.

8. The method of claim 1, wherein the CAX1-like H+/Ca⁺² antiporter polypeptide is SEQ ID NO:4.

9. The method of claim 1, wherein the CAX1-like H+/Ca⁺² antiporter polypeptide is a polypeptide consisting essentially of SEQ ID NO:4.

10. The method of claim 1, wherein the CAX1-like H+/Ca⁺² antiporter polypeptide is a polypeptide selected from the group consisting of:
a) a polypeptide having at least 50% sequence identity with SEQ ID NO:4 and at least 10% of the activity of SEQ ID NO:4;
b) a polypeptide comprising at least 50 consecutive amino acids of SEQ ID NO:4 and having at least 10% of the activity of SEQ ID NO:4; and
c) a polypeptide comprising at least 50 amino acids having at least 50% sequence identity with at least 50 consecutive amino acids of SEQ ID NO:4 and having at least 10% of the activity of SEQ ID NO:4.

11. A method for identifying a compound as a candidate for a herbicide, comprising:
a) measuring the activity of a CAX1-like H+/Ca⁺² antiporter in the presence and absence of a compound, wherein an alteration of the CAX1-like H+/Ca⁺² antiporter activity in the presence of the compound indicates the compound as a candidate for a herbicide.

12. The method of claim 11, wherein the CAX1-like H+/Ca⁺² antiporter is a plant CAX1-like H+/Ca⁺² antiporter.

13. The method of claim 12, wherein the plant is a dicot.

14. The method of claim 12, wherein the plant is a monocot.

15. The method of claim 12, wherein the plant is other than a C3 plant.

16. The method of claim 12, wherein the plant is other than a C4 plant.

17. The method of claim 11, wherein the CAX1-like H+/Ca⁺² antiporter is an Arabidopsis CAX1-like H+/Ca⁺² antiporter.

18. The method of claim 11, wherein the CAX1-like H+/Ca⁺² antiporter is SEQ ID NO:4.

19. The method of claim 11, wherein the CAX1-like H+/Ca⁺² antiporter consists essentially of SEQ ID NO:4.

20. The method of claim 11, wherein the CAX1-like H+/Ca⁺² antiporter is a polypeptide selected from the group consisting of:
a) a polypeptide having at least 50% sequence identity with SEQ ID NO:4 and at least 10% of the activity of SEQ ID NO:4;
b) a polypeptide comprising at least 50 consecutive amino acids of SEQ ID NO:4 and having at least 10% of the activity of SEQ ID NO:4; and
c) a polypeptide comprising at least 50 amino acids having at least 50% sequence identity with at least 50 consecutive amino acids of SEQ ID NO:4 and having at least 10% of the activity of SEQ ID NO:4.

21. A method for identifying a compound as a candidate for a herbicide, comprising:
a) measuring the expression of a CAX1-like H+/Ca⁺² antiporter in a plant, or tissue thereof, in the presence and absence of a compound; and
b) comparing the expression of the CAX1-like H+/Ca⁺² antiporter in the presence and absence of the compound, wherein an altered expression in the presence of the compound indicates that the compound is a candidate for a herbicide.

22. The method of claim 21, wherein the plant is Arabidopsis.

23. The method of claim 21, wherein the expression of the CAX1-like H+/Ca⁺² antiporter is measured by detecting the CAX1-like H+/Ca⁺² antiporter mRNA.

24. The method of claim 21, wherein the expression of the CAX1-like H+/Ca⁺² antiporter is measured by detecting the CAX1-like H+/Ca⁺² antiporter polypeptide.

25. The method of claim 21, wherein the expression of the CAX1-like H+/Ca⁺² antiporter is measured by detecting the CAX1-like H+/Ca⁺² antiporter polypeptide enzyme activity.

26. An isolated nucleic acid comprising a nucleotide sequence that encodes the polypeptide of SEQ ID NO:4.

27. An isolated nucleic acid comprising a nucleotide sequence that encodes a polypeptide consisting essentially of SEQ ID NO:4.

28. A recombinant polypeptide consisting essentially of the amino acid sequence of SEQ ID NO:4.

29. A recombinant polypeptide comprising the amino acid sequence of SEQ ID NO:4.
